(19) United States
(12) Patent Application Publication
(10) Pub. No.: US 2018/0193273 A1
(43) Pub. Date: Jul. 12, 2018

(54) PHARMACEUTICAL COMPOSITION COMPRISING AMLODIPINE AND DEXTROMETHORPHAN

(71) Applicants: TSH BIOPHARM CORPORATION LTD., Taipei City (TW); HANDA PHARMACEUTICALS, INC., Taipei City (TW)

(72) Inventors: Kuo-Chun SUNG, Tainan City (TW); Chun-Liang CHEN, Taipei City (TW); Pei CHEN, Taipei City (TW); Chi-Cheng LIN, Tainan City (TW)

(21) Appl. No.: 15/899,611

(22) Filed: Feb. 20, 2018

Related U.S. Application Data
(63) Continuation of application No. PCT/CN2016/096028, filed on Aug. 19, 2016.
(60) Provisional application No. 62/207,555, filed on Aug. 20, 2015.

(51) Int. Cl. A61K 9/20; A61K 31/4418; A61K 31/485; A61K 47/02; A61K 47/14; A61K 47/38; A61K 9/48; A61P 9/12

(52) U.S. Cl. CPC A61K 9/2009; A61K 31/4418; A61K 31/485; A61K 47/02; A61K 47/14; A61K 2300/00; A61K 9/2054; A61K 9/2059; A61K 9/48; A61P 9/12; A61K 47/38

(57) ABSTRACT

This application provides an oral pharmaceutical composition in a solid form comprising amlodipine or a pharmaceutically acceptable salt thereof, a low dose range of dextromethorphan or a pharmaceutically acceptable salt thereof, and one or more pharmaceutically acceptable excipients. The composition is useful for treating hypertension.

This application is a continuation of PCT/CN2016/096028, filed Aug. 19, 2016; which claims the benefit of U.S. Provisional Application No. 62/207,555, filed Aug. 20, 2015. The contents of the above-identified applications are incorporated herein by reference in their entirety.

### TECHNICAL FIELD

This invention relates to a pharmaceutical composition comprising amlodipine, dextromethorphan, and one or more suitable excipients. The composition is useful for treating hypertension.

### BACKGROUND

Hypertension is a major risk factor for cardiovascular disease and stroke, affects nearly one billion people (about 26% of the adult population) worldwide in 2000, and this is predicted to increase to 1.56 billion by 2025 (Keamey P M, Whelton M, Reynolds K, Muntner P, Whelton P K, He J; Global burden of hypertension: analysis of worldwide data. Lancet 365: 217-23, 2005). Lowering BP significantly reduces the cardiovascular morbidity and mortality (Collins R, Peto R, MacMahon S, et al. Blood pressure, stroke, and coronary heart disease. Part 2, Short-term reductions in blood pressure: overview of randomized drug trials in their epidemiological context. Lancet, 335:827-38, 1990; MacMahon S, Rodgers A, Neal B, et al. Blood pressure lowering for the secondary prevention of myocardial infarction and stroke. Hypertension, 29:537-8, 1997). However, the control rate of hypertension, defined by office BP<140/90 mmHg in non-high-risk patients and <130/80 mmHg in high-risk patients (e.g. patients with diabetes mellitus), is generally low.

It is well known that monotherapy does not provide therapeutic response in all hypertensives. Some patients show an excellent response, while in others there is a poor response. Combination antihypertensive therapy is administered when blood pressure is inadequately controlled by monotherapy to achieve a balanced and additive antihypertensive effect with minimum adverse effects (Cappuccio F P, Macgregor G A. Combination therapy in hypertension. In: Laragh J H, Brenner B M, eds. 2nd Ed. Hypertension: pathophysiology, diagnosis and management. New York: Raven Press, 1995: 2969-83).

Many antihypertensive agents are available in the market. Any of these drugs when used alone as a monotherapy are effective in only 40%-60% of patients with hypertension (Kaplan N. Newer approaches to the treatment of hypertension: part II. Cardiovasc Rev Rep 1979; 8:25-41).

Several studies reported that combination treatment using antihypertensive agents of two different classes are useful and promising in controlling blood pressure in patients with hypertension (Dequattro V. Comparison of benazapril and other antihypertensive agents alone and in combination with the diuretic hydrochlorothiazide. Clin Cardiol 1991; 14:28-32; Brouwer R M L, Bolli P, Eme P. Antihypertensive treatment using calcium antagonists in combination with captopril rather than diuretics. J Cardiovasc Pharmacol 1985; 7:88-91). Calcium channel blockers (CCBs) and ACE inhibitors in combination reduce blood pressure more than either drug alone (Singer D R J. Markandu N D, Shore A C. et al. Captopril and nifedipine in combination for moderate to severe essential hypertension. Hypertension 1987; 9:629-33). Although the combination was more effective than monotherapy in lowering blood pressure, frequent dosing was required for adequate blood pressure control (White N J. Rajagopalan B. Yahaya H, et al. Captopril and frusemide in severe drug resistant hypertension. Lancet 1980; ii: 108-10).

CCB, with the remarkable efficacy in controlling blood pressure and favorable safety profiles, is one of the first-line antihypertensive agents. Amlodipine (AM), a long-acting CCB, is commonly prescribed for the treatment of hypertension. However, in patients who do not respond to lower dose, e.g., 5 mg/day, increasing the dosage to 10˜15 mg/day might lead to peripheral edema, due to potent arterial vasodilatory effects of CCBs. Chen J W, et al. (US2013053411Al) discovered that dextromethorphan (DXM) is effective to lower blood pressure in a subject suffering from hypertension and may acts synergistically with a CCB. Chen J W, et al., also disclosed a combination of a CCB, in particular AM, and DXM for the treatment of hypertension. However, the clinical feasibility of DXM in combination with standard AM treatment remain unknown and need extensive studies.

DXM is a dextrorotatory morphinan and an over-the-counter non-opioid cough suppressant. DXM is a small molecule that can be administered orally, and it has been used clinically for decades with a proven safety record when used at recommended doses (typically 15 to 30 milligrams) (Department of Health and Human Services: National Institutes of Health: Hallucinogens and dissociative drugs including LSD, PCP, ketamine, and DXM. NIH Publication no. 01-2402, March 2001). High-dose chronic use of DXM can lead to the development of toxic psychosis—a mental condition characterized by a loss of contact with reality along with a confused state—as well as other physiological and behavioral problems (Jaffe, J. H. (ed). (1995). Encyclopedia of Drugs and Alcohol, Vol. 1. Simon & Schuster MacMillan: New York).

The development of fixed-dose combinations (FDCs) is becoming increasingly important from a public health perspective. The advantages of fixed-dose combination product include the simplification of therapy, leading to improved compliance product more rapidly effective, higher efficacy or equal efficacy and better safety (World Health Organization. The use of essential drugs. WHO Technical Report Series 825. Geneva: World Health Organization, 1992).

There remains a need of novel effective and safe FDC products, e.g., the combination of AM and DXM, in clinical treatment for treating hypertension.

### SUMMARY OF THE INVENTION

The present invention is directed to a pharmaceutical composition in a solid oral form. The composition comprises amlodipine or a pharmaceutically acceptable salt thereof, dextromethorphan or a pharmaceutically acceptable salt thereof, and one or more pharmaceutically acceptable disintegrants or diluents selected from the group consisting of pregelatinized starch, sodium starch glycolate, microcrystalline cellulose, low-substituted hydroxypropyl cellulose, corn starch, carboxymethylcellulose sodium, croscarmellose sodium, ethylcellulose, talc, dextrin, mannitol, and any combination thereof. The composition optionally comprises a lubricant and/or a glidant.

The present invention also provides a method for treating hypertension by administering an effective amount of the pharmaceutical composition of the present invention to a subject in need.

### BRIEF DESCRIPTION OF THE DRAWINGS

FIG. 1 illustrates the dissolution rate of amlodipine besylate for samples A26-A31.

FIG. 2 illustrates the dissolution rate of dextromethorphan hydrobromide monohydrate for samples A26-A31.

FIG. 3 illustrates the dissolution rate of amlodipine besylate for samples A32-A35.

FIG. 4 illustrates the dissolution rate of dextromethorphan hydrobromide monohydrate for samples A32-A35.

FIG. 5 illustrates the dissolution rate of amlodipine besylate for samples A36-A39.

FIG. 6 illustrates the dissolution rate of dextromethorphan hydrobromide monohydrate for samples A36-A39.

FIG. 7 illustrates the dissolution rate of amlodipine besylate for samples A40-A43.

FIG. 8 illustrates the dissolution rate of dextromethorphan hydrobromide monohydrate for samples A40-A43.

FIG. 9 illustrates the dissolution rate of amlodipine besylate for samples A44-A46.

FIG. 10 illustrates the dissolution rate of dextromethorphan hydrobromide monohydrate for samples A44-A46.

FIG. 11 illustrates the dissolution rate of amlodipine besylate for samples A47-A50.

FIG. 12 illustrates the dissolution rate of dextromethorphan hydrobromide monohydrate for samples A47-A50.

### DETAILED DESCRIPTION OF THE INVENTION

### Reference

### 1. Dissolution

The SUPAC-IR issued on Nov. 30, 1995 by the U.S. FDA adopted the similarity factor f2 proposed by Moore and Flanner (1996) as one criterion for assessing test and reference dissolution similarity

The SUPAC-IR suggests that two dissolution profiles are similar if f2 is between 50 and 100, if >85% dissolution within 15 minutes then the dissolution profile of test and reference are regarded as similar without any further calculation.

The Biopharmaceutics Classification System (BCS) suggests that for high solubility, high permeability drugs and in some instances for high solubility, low permeability drugs, the 85% dissolution in 0.1N HCl within 15 minutes can ensure that the bioavailability of the drug is not limited by dissolution.

### 2. Any Individual Impurity NMT (%)

### ICH Harmonised Tripartite Guideline Impurities in New Drug Products Q3B (R2)

### Thresholds for Degradation Products in New Drug Products

Reporting Thresholds

| Maximum Daily Dose | Threshold |
|---|---|
| ≤1 g | 0.1% |
| >1 g | 0.05% |

Identification Thresholds

| Maximum Daily Dose | Threshold |
|---|---|
| <1 mg | 1.0% or 5 μg TDI, whichever is lower |
| 1 mg-10 mg | 0.5% or 20 μg TDI, whichever is lower |
| >10 mg-2 g | 0.2% or 2 mg TDI, whichever is lower |

### Calculated Value of Identification Thresholds

Identification Thresholds

| | | | Dextromethorphan | | |
|---|---|---|---|---|---|
| Amlodipine besylate | | | hydrobromide monohydrate | | |
| Maximum Daily Dose | | Threshold | Maximum Daily Dose | | Threshold |
| 2.08 | mg | 0.50% | 1.58 | mg | 0.50% |
| 3.47 | mg | 0.50% | 2.5 | mg | 0.50% |
| 3.5 | mg | 0.50% | 2.6 | mg | 0.50% |
| 6.9 | mg | 0.29% | 2.63 | mg | 0.50% |
| 6.94 | mg | 0.29% | 7.5 | mg | 0.27% |
| 13.9 | mg | 0.20% | 7.88 | mg | 0.25% |
| 17.1 | mg | 0.20% | 10 | mg | 0.20% |
| 31.23 | mg | 0.20% | 30 | mg | 0.20% |

| | | | | |
|---|---|---|---|---|
| — | — | 30.7 | mg | 0.20% |

### Definitions

The phrase “pharmaceutically acceptable salt(s)”, as used herein, means those salts of a compound of interest that are safe and effective for pharmaceutical use in mammals and that possess the desired biological activity. Pharmaceutically acceptable salts include salts of acidic or basic groups present in the specified compounds. The acidic or basic groups can be organic or inorganic. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Suitable base salts include, but are not limited to, aluminum, calcium, lithium, magnesium, potassium, sodium, zinc, and diethanolamine salts. Certain compounds used in the present invention can form pharmaceutically acceptable salts with various amino acids, e.g., lysine. N,N′-dibenzylethylenediamine, chloroprocaine, choline, diethanolamine, ethylenediamine, meglumine (N-methylglucamine), procaine, and Tris, and other salts which are currently in widespread pharmaceutical use and are listed in sources well known to those of skill in the art, such as The Merck Index. Any suitable constituent can be selected to make a salt of an active drug discussed herein, provided that it is non-toxic and does not substantially interfere with the desired activity. For a review on pharmaceutically acceptable salts see Berge et al., 66 J. Pharm. Sci 1-19 (1977), which is incorporated herein by reference.

As used herein, “dextromethorphan” or “DXM” refers to the compound (+)-3-methoxy-17-methyl-9α,13α,14α-morphinan, which is also named (+)-3-methoxy-N-methylmorphinan, and any pharmaceutically acceptable salt thereof. For example, DXM can be in a pharmaceutically acceptable salt form selected from the group consisting of salts of free acids, inorganic salts, salts of sulfate, salts of hydrochloride, and salts of hydrobromide. DXM is commercially available as a hydrobromide salt.

DXM is the dextrorotatory (d) enantiomer. Preferably, a pharmaceutical composition according to embodiments of the present invention comprises substantially optically pure DXM or is substantially free of the levorotary (I) enantiomer of DXM.

As used herein, “substantially optically pure DXM” or “substantially free of the levorotary (I) enantiomer of DXM” means that the pharmaceutical composition contains a greater proportion or percentage of DXM in relation to its 1 enantiomer.

DXM can be synthesized and optically purified using methods known in the art, for example as described in U.S. Pat. No. 2,676,177, the content of which is hereby incorporated by reference. It is also available from various commercial sources.

As used herein. “amlodipine” or “AM” refers to the compound 3-ethyl 5-methyl 2-[(2-aminoethoxy)methyl]-4-(2-chlorophenyl)-1,4-dihydro-6-methylpyridine-3,5-dicarboxylate, and any optical isomer, enantiomer, diastereomer, racemate or racemic mixture, pharmaceutically acceptable salts, or pharmaceutically acceptable esters, of the compound. For example, AM can be in a pharmaceutically acceptable salt form of inorganic and organic acids. Such acids are selected from the group consisting of acetic, benzene-sulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. Particularly preferred are besylate, hydrobromic, hydrochloric, phosphoric and sulfuric acids. (See Campbell, S. F. et al., U.S. Pat. No. 4,806,557). AM can also be a pharmaceutically acceptable ester of AM, particularly lower alkyl esters.

AM is a chiral compound. A pharmaceutical composition according to embodiments of the present invention can comprise a racemate, i.e., 1:1 mixture of (R)-(+)- and (S)-(−)-amlodipine or a racemic mixture of the (R)-(+)- and (S)-(−)-amlodipine at different ratios. The pharmaceutical composition can also comprise isolated (R)-(+)-amlodipine or (S)-(−)-amlodipine that is substantially free of the other stereoisomer.

(S)-(−)-amlodipine is a more potent CCB than (R)-(+)-amlodipine. Thus, preferably, a pharmaceutical composition according to embodiments of the present invention comprises substantially optically pure (S)-(−)-amlodipine or is substantially free of (R)-(+)-amlodipine.

As used herein, “substantially optically pure (S)-(−)-amlodipine” or “substantially free of (R)-(+)-amlodipine” means that the pharmaceutical composition contains a greater proportion or percentage of (S)-(−)-amlodipine in relation to (R)-(+)-amlodipine.

The chemical synthesis of the racemic mixture of AM can be performed using methods known in the art, e.g., as described in Arrowsmith. J. E. et al., J. Med. Chem., 29: 1696-1702 (1986). It is also available from various commercial sources. Separation of the AM isomers from the racemic mixture can be performed by methods known in the art, such as those illustrated in U.S. Pat. No. 6,448,275 or U.S. Pat. No. 7,482,464. The contents of the references are hereby incorporated by reference.

As used herein, the term “disintegrant” means a substance which aids dispersion of the tablet in the aqueous medium or gastrointestinal tract, releasing the active ingredient and increasing the surface area for dissolution. Common disintegrants include pregelatinized starch, sodium starch glycolate, crospovidone, alginic acid, sodium alginate, microcrystalline cellulose, powdered cellulose, colloidal silicon dioxide, guar gum, low-substituted hydroxypropyl cellulose, methylcellulose, magnesium aluminum silicate, Croscarmellose sodium, carboxymethylcellulose sodium, carboxymethylcellulose calcium, and starch.

As used herein, the term “diluent” means a substance in a medicinal preparation that lacks pharmacologic activity but is pharmaceutically necessary or desirable. In tablet or capsule dosage forms, it is particularly useful in increasing the bulk of potent drug substances with a mass too small for dosage to allow manufacture or administration. Common diluents include calcium carbonate, calcium lactate, calcium phosphate, calcium silicate, calcium sulfate, cellulose acetate, compressible sugar, corn starch, pregelatinized starch, dextrates, dextrin, dextrose, ethylcellulose, fructose, fumaric acid, kaolin, lactitol, lactose, microcrystalline cellulose, magnesium carbonate, magnesium oxide, maltose, mannitol, polydextrose, polymethacrylates, sodium chloride, sorbitol, sucrose, talc, trehalose, xylitol.

As used herein, the term “lubricant” means a substance which reduces inter-particular friction, prevent adhesion of tablet material to the surface of dies and punches facilitate easy ejection of tablet from die cavity and improve the rate of flow tablet granulation. Common lubricants include calcium stearate, glycerin monostearate, hydrogenated castor oil, hydrogenated vegetable oil type I, magnesium lauryl sulfate, magnesium stearate, poloxamer, polyethylene glycol, sodium lauryl sulfate, sodium stearyl fumarate, stearic acid, talc, zinc stearate.

As used herein, the term “glidant” means a substance which improves flow characteristics of powder mixture. Common glidants include cellulose, powdered, colloidal silicon dioxide, hydrophobic colloidal silica, magnesium oxide, magnesium silicate, magnesium trisilicate, silicon dioxide, talc.

As used herein, the term “subject” means any animal, preferably a mammal, most preferably a human, to whom will be or has been administered compounds or pharmaceutical compositions according to embodiments of the invention. The term “mammal” as used herein, encompasses any mammal. Examples of mammals include, but are not limited to, cows, horses, sheep, pigs, cats, dogs, mice, rats, rabbits, guinea pigs, monkeys, humans etc., more preferably, a human. Preferably, a subject is in need of, or has been the object of observation or experiment of, treatment or prevention of hypertension and symptoms associated therewith.

As used herein, “treating hypertension” means to elicit an antihypertensive effect, such as by providing a normalization to otherwise elevated systolic and/or diastolic blood pressure.

In one embodiment, “treating” refers to an amelioration, prophylaxis, or reversal of a disease or disorder, or at least one discernible symptom thereof, for example, treating hypertension by lowering the elevated systolic and/or diastolic blood pressure.

In another embodiment, “treating” refers to an amelioration, prophylaxis, or reversal of at least one measurable physical parameter related to the disease or disorder being treated, not necessarily discernible symptom in or by the mammal, for example, treating hypertension by decreasing ROS in the vessels.

In yet another embodiment, “treating” refers to inhibiting or slowing the progression of a disease or disorder, either physically, e.g., stabilization of a discernible symptom, physiologically. e.g., stabilization of a physical parameter, or both.

As used herein, the term “effective amount” of a compound refers to the amount of the compound that elicits the effective biological or medicinal response. In a preferred embodiment, the effective amount of a compound is sufficient to treat, improve the treatment of, or prophylactically prevent, hypertension, but is insufficient to cause significant adverse effects associated with administration of the compound.

As used herein, the term “low dose” refers to a dose that is below the lower limit of a standard dose range of a drug when used clinically for treating a disease. For example, the standard dose range of DXM when used clinically is from 10 mg to 60 mg/day. Thus, a low dose of DXM may range from 1 to 10 mg/day. According to the present disclosure. DXM is combined with AM at a dose ranging from 1 to 10 mg/day, preferably from 2.5 to 7.5 mg.

### Description

The present disclosure relates to a pharmaceutical composition comprising AM and a low dose range of DXM and a pharmaceutically acceptable excipient.

Excipients play an important role in formulating a dosage form. These are the ingredients which along with active pharmaceutical ingredients (APIs) make up the dosage forms. However, unfavorable combinations of drug-drug and drug-excipient may result in interaction, which leads to physical instability or chemical instability. Physical instability refers to changes in the characteristics of a drug that do not involve chemical bond formation or breakage in the drug structure, which can be identified by changes in the organoleptic parameters such as appearance, form etc. Chemical instability refers to changes in the chemical structure of the drug molecule resulting in drug degradation, reduced drug content and formation of other molecule such as degradation products. Both physical and chemical instability may cause safety concerns.

In a general aspect, the present invention relates to an oral pharmaceutical composition comprising AM or a pharmaceutically acceptable salt thereof, a low dose range of DXM or a pharmaceutically acceptable salt thereof, one or more disintegrant or a diluents. In one embodiment, the oral pharmaceutical composition further comprises a lubricant and a glidant. In a preferred embodiment, a substantially optically pure DXM, such as a substantially optically pure DXM hydrobromide, is used in the present invention. A combination of AM with a low dose range of DXM improves blood pressure (BP) reduction in a clinical treatment. The oral pharmaceutical composition of the present invention, which comprises AM, a low dose range of DXM and specific excipients, is effective and safe for treating hypertension.

The inventors have discovered that only certain excipients are desirable for a solid oral form of a pharmaceutical composition comprising amlodipine and dextromethorphan. Preferably DXM is in a low dose. The present invention is directed to a pharmaceutical composition in a solid oral form comprising: amlodipine or a pharmaceutically acceptable salt thereof, dextromethorphan or a pharmaceutically acceptable salt thereof, and a pharmaceutically acceptable disintegrant or diluent selected from the group consisting of pregelatinized starch, sodium starch glycolate, microcrystalline cellulose, low-substituted hydroxypropyl cellulose, corn starch, carboxymethylcellulose sodium, croscarmellose sodium, ethylcellulose, talc, dextrin, mannitol, and any combination thereof.

The solid oral form of the pharmaceutical composition of the present invention, which comprises AM, DXM, and the above listed excipient(s), provides acceptable potency of both AM and DXM, i.e., between 90-110% potency according to USP guideline of AM, and provides an acceptable any individual impurity according to ICH guideline Q3B(R2), and provides an acceptable total impurity of not more than (NMT) 1% according to USP35 guideline of AM. The solid oral form of the pharmaceutical composition of the present invention, which comprises AM, DXM, and the above listed excipient(s), also provides a good stability and consistent dissolution behaviors.

Suitable disintegrants or diluents useful for the present invention include pregelatinized starch, sodium starch glycolate, microcrystalline cellulose, powdered cellulose, colloidal silicon dioxide, low-substituted hydroxypropyl cellulose, methylcellulose, carboxymethylcellulose sodium, carboxymethylcellulose calcium, corn starch, dextrin, ethylcellulose, mannitol, and talc.

In one embodiment of the present invention, the disintegrant or diluent is used in an amount ranging from about 1-99%, 2-98%, by weight based on the total weight of the composition.

Examples of the lubricant that can be used in the present application include, but are not limited to, calcium stearate, glycerin monostearate, hydrogenated castor oil, hydrogenated vegetable oil type I, magnesium lauryl sulfate, magnesium stearate, poloxamer, polyethylene glycol, sodium lauryl sulfate, sodium stearyl fumarate, stearic acid, talc, zinc stearate. In a preferred embodiment, the lubricant is magnesium stearate.

Examples of the glidant that can be used in the present application include, but are not limited to, cellulose, powdered, colloidal silicon dioxide, hydrophobic colloidal silica, magnesium oxide, magnesium silicate, magnesium trisilicate, silicon dioxide, and talc. In a preferred embodiment, the glidant is colloidal silicon dioxide.

In one embodiment of the present invention, AM is in an amount of 0.1-30% or 0.1-10% (w/w) of the pharmaceutical composition, and DXM is in an amount of 0.1-30% or 0.1-10% (w/w) of the pharmaceutical composition.

In an embodiment of the present invention, the AM and DXM are administered in a weight ratios of AM versus DXM within about 0.1 to 6.5, or about 0.1 to 4, e.g., about 0.1, 0.2, 0.3, 0.4, 0.5, 0.6, 0.7, 0.8, 0.9, 1, 2, 3, 4, etc., in an oral pharmaceutical composition.

In one embodiment, the unit dosage form is present in an weight ranging from 80 to 1500 mg, preferably from 80 to 1100, more preferably from 80 to 750. e.g., 80, 350, 750, 1100, 1500, etc., in an oral pharmaceutical composition.

In one embodiment of the present invention. DXM can be in a pharmaceutically acceptable salt form selected from the group consisting of salts of free acids, inorganic salts, salts of sulfate, salts of hydrochloride, and salts of hydrobromide. In a preferred embodiment, DXM is in the form of the hydrobromide salt.

In one embodiment of the present invention, AM can be in a pharmaceutically acceptable salt form of inorganic and organic acids. Such acids are selected from the group consisting of acetic, benzene-sulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. In a preferred embodiment, AM is in the form of besylate salt.

The solid oral pharmaceutical composition of the present invention includes pills, tablets, caplets, and hard or soft capsules; including immediate release, timed release, and sustained release formulations, as well as lozenges and dispersible powders or granules. In a preferred embodiment of the present invention, the oral pharmaceutical composition is in the form of a tablet or a capsule. Any of these dosage forms may be prepared according to any method or compounding technique known in the art for the manufacture of pharmaceutical compositions.

In one embodiment of the present invention, an oral pharmaceutical composition comprises a) a therapeutically effective amount ranging from 0.1 to 10% (% w/w) of AM, or a pharmaceutically acceptable salt thereof, b) a therapeutically effective amount ranging from 0.1 to 10% (% w/w) of DXM, or a pharmaceutically acceptable salt thereof, c) a disintegrant or a diluent in an amount of 3 to 90% (% w/w), d) a lubricant in an amount of 0.1 to 3% (% w/w), and e) a glidant in an amount of 0.1 to 3% (% w/w). In one preferred embodiment, the disintegrant or the diluent is pregelatinized starch and/or microcrystalline cellulose, the lubricant is magnesium stearate and the glidant is colloidal silicon dioxide. In one preferred embodiment, the solid oral form comprises 0.1-15% w/w of amlodipine, 0.1-15% w/w of dextromethorphan, 3-60% w/w of pregelatinized starch, and 30-90% w/w microcrystalline cellulose.

In a further aspect, the present disclosure relates to a method for treating hypertension comprising administering to a subject in need thereof an effective amount of the pharmaceutical composition of the present invention.

### EXAMPLES

### Example 1: Compatibility Study

Drug/excipient compatibility considerations and practical studies are to delineate, as quickly as possible, real and possible interactions between potential formulation excipients and the API. This is an important risk reduction exercise early in formulation development.

In the typical drug/excipient compatibility testing program, binary (1:1 or customized) powder mixes are prepared by triturating API with the individual excipients. These powder samples, usually with or without added water and occasionally compacted or prepared as slurries, are stored under accelerated conditions an analyzed by stability-indicating methodology, e.g. HPLC (World Health Organization. Who expert committee on specifications for pharmaceutical preparations. WHO Technical Report Series 929. Geneva: World Health Organization, 2005; Drug-Drug/Drug-Excipient Compatibility Studies on Curcumin using Non-Thermal Methods Advanced Pharmaceutical Bulletin, 2014, 4(3), 309-312). Alternatively, binary samples can be screened using thermal methods, such as DSC/ITC (Compatibility studies of camptothecin with various pharmaceutical excipients used in the development of nanoparticle formulation, Int J Pharm Sci, Vol 5, Suppl 4, 315-321).

The glass scintillation vial samples from Table 1 to 9 were stored at 60° C./75% RH (relative humidity) for 2 weeks and tested for their potency and impurity (Table 10). The compatibility samples obtained in Table 1 to 9 were each subjected to a drug potency and total impurity test under the following conditions.

TABLE 1

| Ingredient | Quantity (mg) | Percentage (%) |
|---|---|---|
| Amlodipine besylate | 7.1 | 100.0 |

Place amlodipine besylate in 20 mL of glass scintillation vial and close screw cap.

TABLE 2

| Ingredient | Quantity (mg) | Percentage (%) |
|---|---|---|
| Amlodipine besylate | 6.8 | 52.7 |
| Dextromethorphan hydrobromide | 6.1 | 47.3 |

Mixing amlodipine besylate and dextromethorphan hydrobromide. Place above mix in 20 mL of glass scintillation vial and close screw cap.

TABLE 3

| Ingredient | Quantity (mg) | Percentage (%) |
|---|---|---|
| Amlodipine besylate | 6.8 | 3.4 |
| Dextromethorphan hydrobromide | 6.3 | 3.2 |
| Pregelatinized Starch | 186.6 | 93.4 |

Mixing amlodipine besylate, dextromethorphan hydrobromide and pregelatinized starch. Place above mix in 20 mL of glass scintillation vial and close screw cap.

TABLE 4

| Ingredient | Quantity (mg) | Percentage (%) |
|---|---|---|
| Amlodipine besylate | 7.2 | 3.6 |
| Dextromethorphan hydrobromide | 6.4 | 3.2 |
| Microcrystalline cellulose (pH 102) | 186.0 | 93.2 |

Mixing amlodipine besylate, dextromethorphan hydrobromide and microcrystalline cellulose.
Place above mix in 20 mL of glass scintillation vial and close screw cap.

TABLE 5

| Ingredient | Quantity (mg) | Percentage (%) |
|---|---|---|
| Amlodipine besylate | 7.2 | 3.7 |
| Dextromethorphan hydrobromide | 6.1 | 3.1 |
| Calcium phosphate dibasic anhydrous | 183.2 | 93.2 |

Mixing amlodipine besylate, dextromethorphan hydrobromide and calcium phosphate dibasic anhydrous. Place above mix in 20 mL of glass scintillation vial and close screw cap.

TABLE 6

| Ingredient | Quantity (mg) | Percentage (%) |
|---|---|---|
| Amlodipine besylate | 7.8 | 20.6 |
| Dextromethorphan hydrobromide | 6.7 | 17.7 |
| Sodium starch glycolate | 23.3 | 61.7 |

Mixing amlodipine besylate, dextromethorphan hydrobromide and sodium starch glycolate.
Place above mix in 20 mL of glass scintillation vial and close screw cap.

TABLE 7

| Ingredient | Quantity (mg) | Percentage (%) |
|---|---|---|
| Amlodipine besylate | 6.9 | 20.5 |
| Dextromethorphan hydrobromide | 6.8 | 20.3 |
| Crospovidone XL | 19.9 | 59.2 |

Mixing amlodipine besylate, dextromethorphan hydrobromide and crospovidone XL. Place above mix in 20 mL of glass scintillation vial and close screw cap.

TABLE 8

| Ingredient | Quantity (mg) | Percentage (%) |
|---|---|---|
| Amlodipine besylate | 7.1 | 1.8 |
| Dextromethorphan hydrobromide | 6.4 | 1.6 |
| Pregelatinized starch | 187.6 | 48.0 |
| Microcrystalline cellulose (pH 102) | 185.2 | 47.4 |
| Magnesium stearate | 2.1 | 0.6 |
| Colloidal silicon dioxide | 2.1 | 0.6 |

Mixing amlodipine besylate, dextromethorphan hydrobromide, pregelatinized starch, microcrystalline cellulose, magnesium stearate and colloidal silicon dioxide. Place above mix in 20 mL of glass scintillation vial and close screw cap.

TABLE 9

| Ingredient | Quantity (mg) | Percentage (%) |
|---|---|---|
| Amlodipine besylate | 7.1 | 1.8 |
| Dextromethorphan hydrobromide | 6.4 | 1.6 |
| Microcrystalline cellulose (pH 102) | 186.4 | 45.9 |
| Calcium phosphate dibasic anhydrous | 183.8 | 45.2 |
| Magnesium stearate | 3.1 | 0.7 |
| Crospovidone XL | 19.5 | 4.8 |

Mixing amlodipine besylate, dextromethorphan hydrobromide, microcrystalline cellulose calcium phosphate dibasic anhydrous, magnesium stearate and crospovidone XL. Place above mix in 20 mL of glass scintillation vial and close screw cap.

### Analytical Conditions

Column: stainless steel column (inner diameter: 4.6 mm, length: 15 cm) filled with octadecylsilanized silica gel for 5 pun liquid chromatography
Mobile Phase: Methanol, acetonitrile, and Buffer (35:15:50)
Buffer: 0.7% triethylamine with phosphoric acid to a pH of 3.0±0.1.

### Mode: LC

### Detector: UV 237 nm

Flow rate: 1 mL/min
Injection size: 50 μL

The results are as listed in Table 10. As shown in Table 10 the AM compatibility is most stable with dextromethorphan hydrobromide, pregelatinized starch, microcrystalline cellulose, magnesium stearate and colloidal silicon dioxide. These results show that the increase in impurities in the composition is related to, but not directly proportional to loss of potency.

TABLE 10

| Ingredient | Function | Table 1 | Table 2 | Table 3 | Table 4 | Table 5 | Table 6 | Table 7 | Table 8 | Table 9 |
|---|---|---|---|---|---|---|---|---|---|---|
| Amlodipine besylate | Active | 7.1 mg/ | 6.8 mg/ | 6.8 mg/ | 7.2 mg/ | 7.2 mg/ | 7.8 mg/ | 6.9 mg/ | 7.1 mg/ | 7.1 mg/ |
| | | 100% | 52.7% | 3.4% | 3.6% | 3.7% | 20.6% | 20.5% | 1.8% | 1.8% |
| Dextromethorphan | Active | x | 6.1 mg/ | 6.3 mg/ | 6.4 mg/ | 6.1 mg/ | 6.7 mg/ | 6.8 mg/ | 6.4 mg/ | 6.4 mg/ |
| hydrobromide | | | 47.3% | 3.2% | 3.1% | 3.1% | 17.7% | 20.3% | 1.6% | 1.6% |
| Pregelatinized starch | Diluent/ | x | x | 186.6 mg/ | x | x | x | x | 187.6 mg/ | x |
| | Disintegrant | | | 93.4% | | | | | 48.0% | |
| Microcrystalline | Diluent/ | x | x | x | 186.0 mg/ | x | x | x | 185.2 mg/ | 186.4 mg/ |
| cellulose (pH 102) | Disintegrant | | | | 93.2% | | | | 47.4% | 45.9% |
| Calcium phosphate | Diluent | x | x | x | x | 183.2 mg/ | x | x | x | 183.8 mg/ |
| dibasic anhydrous | | | | | | 93.2% | | | | 45.2% |
| Sodium starch | Disintegrant | x | x | x | x | x | 23.3 mg/ | x | x | x |
| glycolate | | | | | | | 61.7% | | | |
| Crospovidone XL | Disintegrant | x | x | x | x | x | x | 19.9 mg/ | x | 19.5 mg/ |
| | | | | | | | | 59.2% | | 4.8% |
| Magnesium stearate | Lubricant | x | x | x | x | x | x | x | 2.1 mg/ | 3.1 mg/ |
| | | | | | | | | | 0.6% | 0.7% |
| Colloidal silicon | Glidant | x | x | x | x | x | x | x | 2.1 mg/ | x |
| dioxide | | | | | | | | | 0.6% | |
| % Potency | x | 100.2 | 99.6 | 97.4 | 99.8 | 80.5 | 90.3 | 95.4 | 96.8 | 88.6 |
| (Amlodipine | | | | | | | | | | |
| besylate) | | | | | | | | | | |
| % Potency | x | x | 98.2 | 99.7 | 97.1 | 101.0 | 93.4 | 98.6 | 103.0 | 99.6 |
| (Dextromethorphan | | | | | | | | | | |
| hydrobromide) | | | | | | | | | | |
| Total impurity | x | 0.02 | 0.05 | 0.03 | 0.00 | 2.44 | 0.20 | 0.48 | 0.05 | 1.02 |

The results show that Table 3, 4, 6, and 8 with excipients of pregalatinized starch Table 3), microcrystalline cellulose (Table 4), sodium starch glycolate (Table 6), and pregalatinized starch, microcrystalline cellulose, magnesium sterate, colloidal silicon dioxide. (Table 8) provide good potency and acceptable impurity. Excipients of Tables 5, 7, and 9 do not provide good potency or acceptable impurity.

### Example 2: Tablet Stability Study

The amlodipine besylate-dextromethorphan hydrobromide combined tablets from Table 11 and Table 12 were stored at 60° C./75% RH (relative humidity) for 2 weeks and tested for their potency and impurity (Table 13). Furthermore, as shown in Table 14, the total impurity of Sample II was not more than 0.26% after 6 months at 40° C./75% RH (relative humidity) condition.

TABLE 11
Sample I

| Ingredient | gm | % |
|---|---|---|
| Amlodipine besylate | 4.86 | 3.5 |
| Dextromethorphan hydrobromide | 4.20 | 3.0 |
| Calcium phosphate dibasic anhydrous | 60.94 | 43.5 |
| Microcrystalline cellulose (pH 102) | 61.60 | 44.0 |
| Magnesium stearate | 1.40 | 1.0 |
| Sodium starch glycolate | 7.00 | 5.0 |

Microcrystalline cellulose, Amlodipine besylate, Dextromethorphan hydrobromide, Calcium phosphate dibasic anhydrous, Sodium starch glycolate were each passed through a #30 mesh and mixed for 3 mins, Subsequently Magnesium stearate was added thereto, mixed for 1 mins, and the resulting mixture was subjected to tableting with an compression hardness of about 10 kgf using a tablet press (Batch scale: 140 gm/700 tablets).

TABLE 12
Sample II

| Ingredient | gm | % |
|---|---|---|
| Amlodipine besylate | 2.78 | 3.0 |
| Dextromethorphan hydrobromide | 3.00 | 3.3 |
| Pregelatinized starch | 40.00 | 43.5 |
| Microcrystalline cellulose (pH 102) | 45.42 | 49.4 |
| Magnesium stearate | 0.4 | 0.4 |
| Colloidal silicon dioxide | 0.4 | 0.4 |

Microcrystalline cellulose, Amlodipine besylate, Dextromethorphan hydrobromide, Pregelatinized starch, Colloidal silicon dioxide were each passed through a #30 mesh and mixed for 3 mins, Subsequently Magnesium stearate was added thereto, mixed for 1 mins, and the resulting mixture was subjected to tableting with an compression hardness of about 10 kgf using a tablet press (Batch scale: 92 gm/400 tablets).

The amlodipine besylate-dextromethorphan hydrobromide combined tablets obtained in Table 11 and 12 were each subjected to a drug potency and total impurity test under the following conditions.

### Analytical Conditions

Column: stainless steel column (inner diameter: 4.6 mm, length: 15 cm) filled with octadecylsilanized silica gel for 5 μm liquid chromatography
Mobile Phase: Methanol, acetonitrile, and Buffer (35:15:50)
Buffer: 0.7% triethylamine with phosphoric acid to a pH of 3.0±0.1.

### Mode: LC

### Detector: UV 237 nm

Flow rate: 1 mL/min
Injection size: 50 μL

The results are as listed in Table 13. As shown in Table 13, the combined tablets exhibited Sample II even lower total impurity than Sample I. As shown in Tablet 14 the combined tablets obtained in Sample II exhibited the resulting product maintains more than 97% of its initial potency with its total impurity not more than 0.26% after 6 months of storage at 40° C./75% RH (relative humidity) condition.

TABLE 13

| Ingredient | Function | Sample I | | Sample II | |
|---|---|---|---|---|---|
| Amlodipine | Active | 6.94 | mg/3.5% | 6.94 | mg/3.0% |
| besylate | | | | | |
| Dextromethorphan | Active | 6.00 | mg/3.0% | 7.50 | mg/3.3% |
| hydrobromide | | | | | |
| Microcrystalline | Diluent/ | 88.00 | mg/44.0% | 113.56 | mg/49.4% |
| cellulose | Disin- | | | | |
| (pH 102) | tegrant | | | | |
| Calcium phosphate | Diluent | 87.06 | mg/43.5% | x | |
| dibasic anhydrous | | | | | |
| Pregelatinized | Diluent/ | x | | 100.00 | mg/43.5% |
| starch | Disin- | | | | |
| | tegrant | | | | |
| Magnesium stearate | Lubricant | 2.00 | mg/1.0% | 100 | mg/0.4% |

| | | | | |
|---|---|---|---|---|
| Sodium starch | Disin- | 10.00 | mg/5.0% | x |
| glycolate | tegrant | | | |

| | | | | |
|---|---|---|---|---|
| Colloidal silicon | Glidant | x | 1.00 | mg/0.4% |
| dioxide | | | | |

| | | | |
|---|---|---|---|
| % Potency | x | 96.3 | 97.9 |
| (Amlodipine | | | |
| besylate) | | | |
| % Potency | x | 103.8 | 99.4 |
| (Dextromethorphan | | | |
| hydrobromide) | | | |
| Total impurity | x | 0.37 | 0.17 |

TABLE 14

| Ingredient | Initial | 1 M | 3 M | 6 M |
|---|---|---|---|---|
| % Potency (Amlodipine besylate) | 100.5 | 99.0 | 98.3 | 97.0 |
| % Potency (Dextromethorphan | 99.3 | 98.0 | 101.9 | 99.9 |
| hydrobromide) | | | | |
| Total impurity | 0.06 | 0.07 | 0.11 | 0.22 |

### Example 3: The Amlodipine Besylate-Dextromethorphan Hydrobromide Combined Tablets

The amlodipine besylate-dextromethorphan hydrobromide combined tablets obtained in Sample A to Sample E of Table 15.

TABLE 15

| Ingredient | Function | Sample A | Sample B | Sample C | Sample D | Sample E |
|---|---|---|---|---|---|---|
| Amlodipine besylate | Active | 8.7% | 2.0% | 0.9% | 0.6% | 0.5% |
| Dextromethorphan | Active | 9.4% | 2.1% | 1.0% | 0.7% | 0.5% |
| hydrobromide | | | | | | |
| Microcrystalline | Diluent/ | 50.0% | 50.0% | 50.0% | 50.0% | 50.0% |
| cellulose (pH 102) | Disintegrant | | | | | |
| Pregelatinized starch | Diluent/ | 31.1% | 45.1% | 47.3% | 47.9% | 48.2% |
| | Disintegrant | | | | | |
| Magnesium stearate | Lubricant | 0.4% | 0.4% | 0.4% | 0.4% | 0.4% |
| Colloidal silicon dioxide | Glidant | 0.4% | 0.4% | 0.4% | 0.4% | 0.4% |
| Total weight | x | 80.0 mg | 350.0 mg | 750.0 mg | 1100.0 mg | 1500.0 mg |

Microcrystalline cellulose, Amlodipine besylate, Dextromethorphan hydrobromide, Pregelatinized starch, Colloidal silicon dioxide were each passed through a #30 mesh and mixed for 3 mins, Subsequently Magnesium stearate was added thereto, mixed for 1 mins, and the resulting mixture was subjected to tableting with an compression hardness of about 10 kgf using a tablet press.

### Example 4. Different Excipients

The glass scintillation vial samples from Sample A1 to A25 were stored at 60° C./75% RH (relative humidity) for 2 weeks and tested for their potency and impurity.
Sample A1. Place amlodipine besylate in 20 mL of glass scintillation vial and close screw cap.
Sample A2 Mixing amlodipine besylate and dextromethorphan hydrobromide monohydrate.

Place above mix in 20 mL of glass scintillation vial and close screw cap.
Sample A3. Mixing amlodipine besylate, dextromethorphan hydrobromide monohydrate and pregelatinized starch. Place above mix in 20 mL of glass scintillation vial and close screw cap.
Sample A4. Mixing amlodipine besylate, dextromethorphan hydrobromide monohydrate and sodium starch glycolate. Place above mix in 20 mL of glass scintillation vial and close screw cap.
Sample A5. Mixing amlodipine besylate, dextromethorphan hydrobromide monohydrate and crospovidone. Place above mix in 20 mL of glass scintillation vial and close screw cap.
Sample A6. Mixing amlodipine besylate, dextromethorphan hydrobromide monohydrate and microcrystalline cellulose. Place above mix in 20 mL of glass scintillation vial and close screw cap.
Sample A7. Mixing amlodipine besylate, dextromethorphan hydrobromide monohydrate and Low-substituted hydroxypropyl cellulose. Place above mix in 20 mL of glass scintillation vial and close screw cap.
Sample A8. Mixing amlodipine besylate, dextromethorphan hydrobromide monohydrate and corn starch. Place above mix in 20 mL of glass scintillation vial and close screw cap.
Sample A9. Mixing amlodipine besylate, dextromethorphan hydrobromide monohydrate and carboxymethylcellulose sodium. Place above mix in 20 mL of glass scintillation vial and close screw cap.
Sample A10. Mixing amlodipine besylate, dextromethorphan hydrobromide monohydrate and croscarmellose sodium. Place above mix in 20 mL of glass scintillation vial and close screw cap.
Sample A11. Mixing amlodipine besylate, dextromethorphan hydrobromide monohydrate and ethylcellulose. Place above mix in 20 mL of glass scintillation vial and close screw cap.
Sample A12. Mixing amlodipine besylate, dextromethorphan hydrobromide monohydrate and calcium phosphate. Place above mix in 20 mL of glass scintillation vial and close screw cap.
Sample A13. Mixing amlodipine besylate, dextromethorphan hydrobromide monohydrate and magnesium oxide. Place above mix in 20 mL of glass scintillation vial and close screw cap.
Sample A14. Mixing amlodipine besylate, dextromethorphan hydrobromide monohydrate and mannitol. Place above mix in 20 mL of glass scintillation vial and close screw cap.
Sample A15. Mixing amlodipine besylate, dextromethorphan hydrobromide monohydrate and talc. Place above mix in 20 mL of glass scintillation vial and close screw cap.
Sample A16. Mixing amlodipine besylate, dextromethorphan hydrobromide monohydrate and lactose. Place above mix in 20 mL of glass scintillation vial and close screw cap.
Sample A17. Mixing amlodipine besylate, dextromethorphan hydrobromide monohydrate and calcium carbonate. Place above mix in 20 mL of glass scintillation vial and close screw cap.
Sample A18. Mixing amlodipine besylate, dextromethorphan hydrobromide monohydrate and fumaric acid. Place above mix in 20 mL of glass scintillation vial and close screw cap.
Sample A19. Mixing amlodipine besylate, dextromethorphan hydrobromide monohydrate and sodium chloride. Place above mix in 20 mL of glass scintillation vial and close screw cap.
Sample A20. Mixing amlodipine besylate, dextromethorphan hydrobromide monohydrate and sucrose. Place above mix in 20 mL of glass scintillation vial and close screw cap.
Sample A21. Mixing amlodipine besylate, dextromethorphan hydrobromide monohydrate and dextrose. Place above mix in 20 mL of glass scintillation vial and close screw cap.
Sample A22. Mixing amlodipine besylate, dextromethorphan hydrobromide monohydrate and calcium sulfate. Place above mix in 20 mL of glass scintillation vial and close screw cap.
Sample A23. Mixing amlodipine besylate, dextromethorphan hydrobromide monohydrate and fructose. Place above mix in 20 mL of glass scintillation vial and close screw cap.
Sample A24. Mixing amlodipine besylate, dextromethorphan hydrobromide monohydrate and dextrin. Place above mix in 20 mL of glass scintillation vial and close screw cap.
Sample A25. Mixing amlodipine besylate, dextromethorphan hydrobromide monohydrate and kaolin. Place above mix in 20 mL of glass scintillation vial and close screw cap.

### Analytical Condition

Column: stainless steel column (inner diameter: 4.6 mm, length: 15 cm) filled with octadecylsilanized silica gel for 5 μm liquid chromatography
Mobile Phase: Methanol, acetonitrile, and Buffer (35:15:50)
Buffer: 0.7% triethylamine with phosphoric acid to a pH of 3.0±0.1.

### Mode: LC

### Detector: UV 237 nm

Flow rate: 1 mL/min
Injection size: 50 μL

Table 16 shows the potency and impurity results of samples A1-A25. The results show that samples A5, A12-A14, A16-23, and A25, which do not have the desired disintegrants or diluents of the present invention, do not provide acceptable potency or impurity results.

### Example 5. AM/DXM Weight Ratio

The amlodipine besylate-dextromethorphan hydrobromide combined tablets from Sample A26 to Sample A31 were each subjected to a drug dissolution test and stored at 60° C./75% RH (relative humidity) for 2 weeks and tested for their potency and impurity.

### Samples Condition

Microcrystalline cellulose, amlodipine besylate, dextromethorphan hydrobromide monohydrate, pregelatinized starch, colloidal silicon dioxide were each passed through a #30 mesh and mixed for 3 mins, Subsequently magnesium stearate was added thereto, mixed for 1 mins, and the resulting mixture was subjected to tableting with an compression using a tablet press.

### Dissolution Condition

### Effluent: 500 mL of 0.01N HCl

Dissolution-test system: USP paddle method, 75 rpm

### Temperature: 37° C.

### Analytical Condition

Column: stainless steel column (inner diameter: 4.6 mm, length: 15 cm) filled with octadecylsilanized silica gel for 5 μm liquid chromatography
Mobile Phase: Methanol, acetonitrile, and Buffer (35:15:50)
Buffer: 0.7% triethylamine with phosphoric acid to a pH of 3.0±0.1.

### Mode: LC

### Detector: UV 237 nm

Flow rate: 1 mL/min
Injection size: 50 μL

Table 17 shows the potency and impurity results of samples A26-A31. The results show that samples A26-A31, which include the weight ratio of AM and DXM from 0.1 to 6.5, provide acceptable potency or impurity results.

### Example 6. Weight Percentage of the Excipient

The amlodipine besylate-dextromethorphan hydrobromide combined tablets from Sample A32 to Sample A43 were each subjected to a drug dissolution test and stored at 60° C./75% RH (relative humidity) for 2 weeks and tested for their potency and impurity.

### Samples Condition

Microcrystalline cellulose (mannitol), amlodipine besylate, dextromethorphan hydrobromide monohydrate, pregelatinized starch, colloidal silicon dioxide were each passed through a #30 mesh and mixed for 3 mins, Subsequently magnesium stearate was added thereto, mixed for 1 mins, and the resulting mixture was subjected to tableting with an compression using a tablet press.

### Dissolution Condition

### Effluent: 500 mL of 0.01N HCl

Dissolution-test system: USP paddle method, 75 rpm

### Temperature: 37° C.

### Analytical Condition

Column: stainless steel column (inner diameter: 4.6 mm, length: 15 cm) filled with octadecylsilanized silica gel for 5 μm liquid chromatography
Mobile Phase: Methanol, acetonitrile, and Buffer (35:15:50)
Buffer: 0.7% triethylamine with phosphoric acid to a pH of 3.0±0.1.

### Mode: LC

### Detector: UV 237 nm

Flow rate: 1 mL/min
Injection size: 50 μL

Tables 18-20 show the potency and impurity results of samples A32-A43. The results show that samples A32-A43, which include the weight ratio of the desired disintegrants or diluents of the present invention from 0.1% to 98%, provide acceptable potency or impurity results.

### Example 7. Weight of the Composition

The amlodipine besylate-dextromethorphan hydrobromide combined tablets from Sample A44 to Sample A46 were each subjected to a drug dissolution test and stored at 60° C./75% RH (relative humidity) for 2 weeks and tested for their potency and impurity.

### Samples Condition

Microcrystalline cellulose, amlodipine besylate, dextromethorphan hydrobromide monohydrate, pregelatinized starch, colloidal silicon dioxide were each passed through a #30 mesh and mixed for 3 mins, Subsequently magnesium stearate was added thereto, mixed for 1 mins, and the resulting mixture was subjected to tableting with an compression using a tablet press.

### Dissolution Condition

### Effluent: 500 mL of 0.01N HCl

Dissolution-test system: USP paddle method, 75 rpm

### Temperature: 37° C.

### Analytical Condition

Column: stainless steel column (inner diameter: 4.6 mm, length: 15 cm) filled with octadecylsilanized silica gel for 5 μm liquid chromatography
Mobile Phase: Methanol, acetonitrile, and Buffer (35:15:50)
Buffer: 0.7%0/triethylamine with phosphoric acid to a pH of 3.0±0.1.

### Mode: LC

### Detector: UV 237 nm

Flow rate: 1 mL/min
Injection size: 50 μL

Table 21 shows the potency and impurity results of samples A44-A46. The results show that samples A44-A46, which weight of the composition of the present invention from 80 mg to 1500 mg, provide acceptable potency or impurity results.

### Example 8. Formulation Examples

The amlodipine besylate-dextromethorphan hydrobromide combined tablets from Sample A47 to Sample A50 were each subjected to a drug dissolution test and stored at 60° C./75% RH (relative humidity) for 2 weeks and tested for their potency and impurity.

### Samples Condition

Microcrystalline cellulose, amlodipine besylate, dextromethorphan hydrobromide monohydrate, pregelatinized starch, colloidal silicon dioxide were each passed through a #30 mesh and mixed for 3 mins, Subsequently magnesium stearate was added thereto, mixed for 1 mins, and the resulting mixture was subjected to tableting with an compression using a tablet press.

### Dissolution Condition

### Effluent: 500 mL of 0.01N HCl

Dissolution-test system: USP paddle method, 75 rpm

### Temperature: 37° C.

### Analytical Condition

Column: stainless steel column (inner diameter: 4.6 mm, length: 15 cm) filled with octadecylsilanized silica gel for 5 m liquid chromatography
Mobile Phase: Methanol, acetonitrile, and Buffer (35:15:50)
Buffer: 0.7% triethylamine with phosphoric acid to a pH of 3.0±0.1.

### Mode: LC

### Detector: UV 237 nm

Flow rate: 1 mL/min
Injection size: 50 μL

Table 22 shows the potency and impurity results of samples A47-A50. The results show that samples A47-A50, which include the desired excipients of the present invention, provide acceptable potency or impurity results.

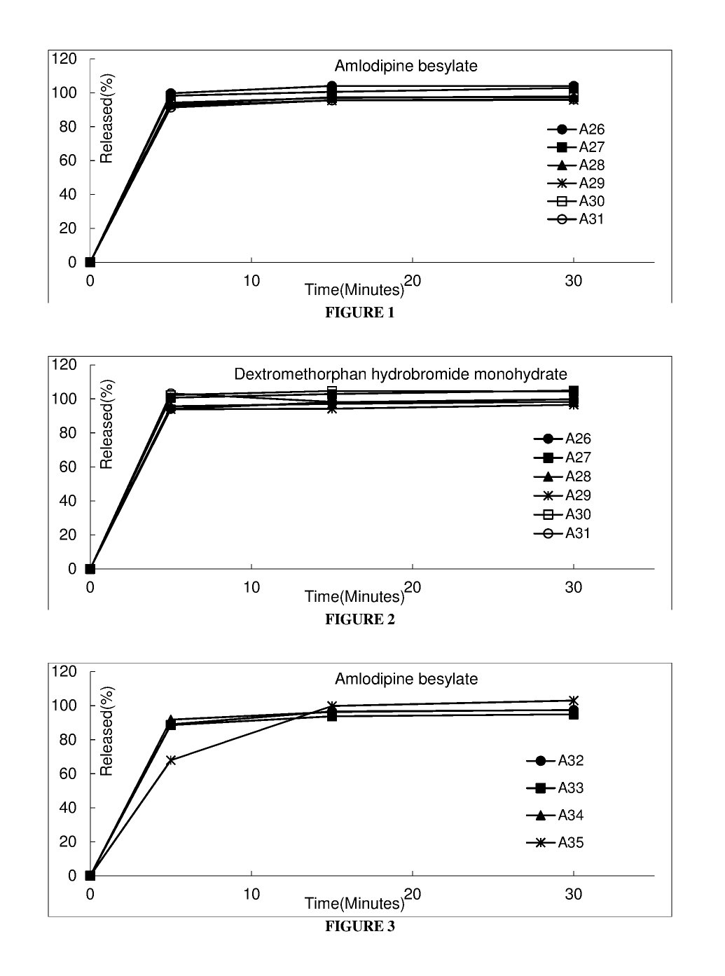

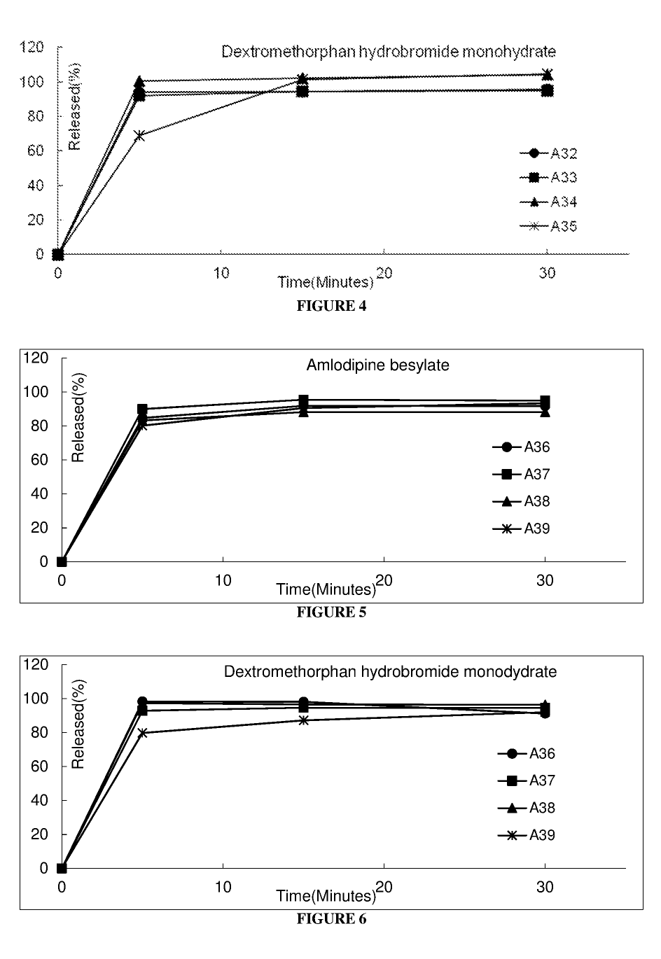

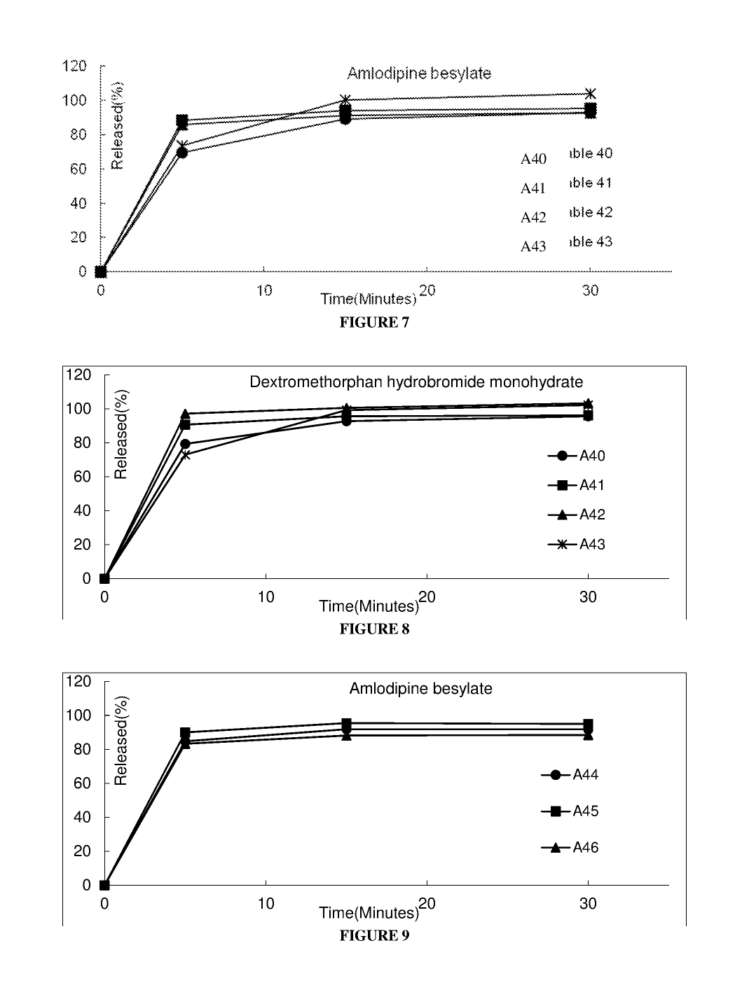

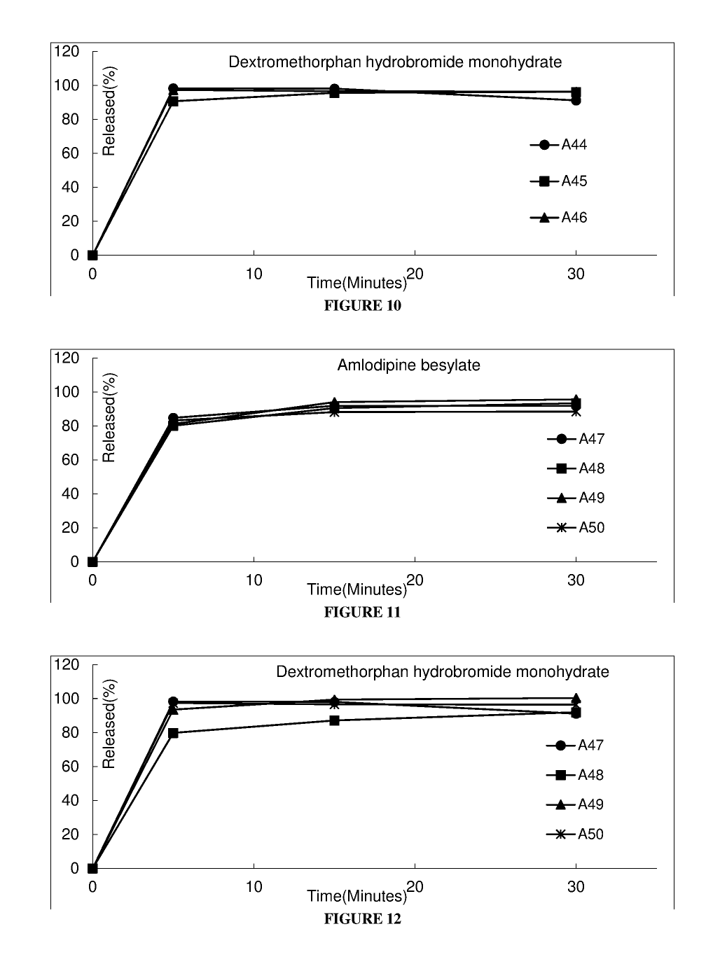

FIGS. 1-12 illustrates the dissolution rate of amlodipine besylate and the dissolution rate of dextromethorphan hydrobromide monohydrate for samples A26-A50.

Since the dissolution of AM and DXM in each formulation are >85% within 15 minutes, the results indicate that dissolution profiles of the various formulation are similar.

TABLE 16

| | Sample | | Sample | | Sample | | Sample | | Sample | | Sample | |
|---|---|---|---|---|---|---|---|---|---|---|---|---|
| | A1 | | A2 | | A3 | | A4 | | A5 | | A6 | |
| | mg | % | mg | % | mg | % | mg | % | mg | % | mg | % |
| Amlodipine besylate | 6.9 | 100.0 | 6.9 | 47.9 | 6.9 | 2.6 | 6.9 | 20.0 | 6.9 | 26.1 | 6.9 | 2.6 |
| Dextromethorphan hydrobromide monohydrate | — | — | 7.5 | 52.1 | 7.5 | 2.8 | 7.5 | 21.8 | 7.5 | 28.4 | 7.5 | 2.8 |
| Pregelatinized starch | — | — | — | — | 250.0 | 94.6 | — | — | — | — | — | — |
| Sodium starch glycolate | — | — | — | — | — | — | 20.0 | 58.2 | — | — | — | — |
| Crospovidone | — | — | — | — | — | — | | | 12.0 | 45.5 | — | — |
| Microcrystalline cellulose | — | — | — | — | — | — | — | — | — | — | 250.0 | 94.6 |
| % Potency (Amlodipine besylate): 90%~10% | — | 100.2 | — | 99.6 | — | 97.6 | — | 95.7 | — | 96.7 | — | 97.8 |
| % Potency (Dextromethorphan hydrobromide | — | — | — | 98.2 | — | 97.5 | — | 100.4 | — | 102.6 | — | 98.7 |
| monohydrate): 90%~110% | | | | | | | | | | | | |
| Any individual impurity (%): NMT 0.26% | — | 0.02 | — | 0.05 | — | 0.05 | — | 0.15 | — | 0.32 | — | 0.09 |
| Based on ICH Q3B (R2) | | | | | | | | | | | | |
| identification thresholds | | | | | | | | | | | | |
| Total impurity (%): NMT 1.00% | — | 0.02 | — | 0.05 | — | 0.05 | — | 0.15 | — | 0.50 | — | 0.09 |

| | Sample | | Sample | | Sample | | Sample | |
|---|---|---|---|---|---|---|---|---|
| | A7 | | A8 | | A9 | | A10 | |
| | mg | % | mg | % | mg | % | mg | % |
| Amlodipine besylate | 6.9 | 9.3 | 6.9 | 2.6 | 6.9 | 23.5 | 6.9 | 26.1 |
| Dextromethorphan hydrobromide monohydrate | 7.5 | 10.1 | 7.5 | 2.8 | 7.5 | 25.5 | 7.5 | 28.4 |
| Low-substituted hydroxypropyl cellulose | 60.0 | 80.6 | — | — | — | — | — | — |
| Corn starch | — | — | 250.0 | 94.6 | — | — | — | — |
| Carboxymethylcellulose sodium | — | — | — | — | 15.0 | 51.0 | — | — |
| Croscarmellose sodium | — | — | — | — | — | — | 12.0 | 45.5 |
| % Potency (Amlodipine besylate): 90%~110% | — | 93.2 | — | 99.6 | — | 96.2 | — | 97.7 |
| % Potency (Dextromethorphan hydrobromide monohydrate): 90%~110% | — | 100.0 | — | 99.2 | — | 95.4 | — | 98.6 |
| Any individual impurity (%): NMT 0.26% | — | 0.09 | — | 0.03 | — | 0.23 | — | N.D. |
| Based on ICH Q3B (R2) identification thresholds | | | | | | | | |
| Total impurity (%): NMT 1.00% | — | 0.09 | — | 0.05 | — | 0.32 | — | N.D. |

| | Sample | | Sample | | Sample | | Sample | | Sample | | Sample | | Sample | |
|---|---|---|---|---|---|---|---|---|---|---|---|---|---|---|
| | A11 | | A12 | | A13 | | A14 | | A15 | | A16 | | A17 | |
| | mg | % | mg | % | mg | % | mg | % | mg | % | mg | % | mg | % |
| Amlodipine besylate | 6.9 | 2.6 | 6.9 | 2.6 | 6.9 | 2.6 | 6.9 | 2.6 | 6.9 | 2.6 | 6.9 | 2.6 | 6.9 | 2.6 |
| Dextromethorphan hydrobromide | 7.5 | 2.8 | 7.5 | 2.8 | 7.5 | 2.8 | 7.5 | 2.8 | 7.5 | 2.8 | 7.5 | 2.8 | 7.5 | 2.8 |
| monohydrate | | | | | | | | | | | | | | |
| Ethylcellulose | 250.0 | 94.6 | — | — | — | — | — | — | — | — | — | — | — | — |
| Calcium phosphate | — | — | 250.0 | 94.6 | — | — | — | — | — | — | — | — | — | — |
| Magnesium oxide | — | — | — | — | 250.0 | 94.6 | — | — | — | — | — | — | — | — |
| Mannitol | — | — | — | — | — | — | 250.0 | 94.6 | — | — | — | — | — | — |
| Talc | — | — | — | — | — | — | — | — | 250.0 | 94.6 | — | — | — | — |
| Lactose | — | — | — | — | — | — | — | — | — | — | 250.0 | 94.6 | — | — |
| Calcium carbonate | — | — | — | — | — | — | — | — | — | — | — | — | 250.0 | 94.6 |
| % Potency (Amlodipine besylate): | — | 97.7 | — | 79.4 | — | 61.7 | — | 98.0 | — | 93.8 | — | 94.4 | — | 77.5 |
| 90%~110% | | | | | | | | | | | | | | |
| % Potency (Dextromethorphan | — | 106.7 | — | 108.2 | — | 74.2 | — | 103.6 | — | 100.5 | — | 99.3 | — | 92.5 |
| hydrobromide monohydrate): | | | | | | | | | | | | | | |
| 90%~110% | | | | | | | | | | | | | | |
| Any individual impurity (%): NMT | — | 0.07 | — | 2.96 | — | 4.36 | — | 0.30 | — | 0.14 | — | 0.94 | — | 2.62 |
| 0.26% Based on ICH Q3B | | | | | | | | | | | | | | |
| (R2) identification | | | | | | | | | | | | | | |
| thresholds | | | | | | | | | | | | | | |
| Total impurity (%): NMT 1.00% | — | 0.11 | — | 3.56 | — | 6.87 | — | 0.30 | — | 0.27 | — | 1.39 | — | 3.41 |

| | Sample | | Sample | | Sample | | Sample | | Sample | | Sample | |
|---|---|---|---|---|---|---|---|---|---|---|---|---|
| | A18 | | A19 | | A20 | | A21 | | A22 | | A23 | |
| | mg | % | mg | % | mg | % | mg | % | mg | % | mg | % |
| Amlodipine besylate | 6.9 | 2.6 | 6.9 | 2.6 | 6.9 | 2.6 | 6.9 | 2.6 | 6.9 | 2.6 | 6.9 | 2.6 |
| Dextromethorphan hydrobromide monohydrate | 7.5 | 2.8 | 7.5 | 2.8 | 7.5 | 2.8 | 7.5 | 2.8 | 7.5 | 2.8 | 7.5 | 2.8 |
| Fumaric acid | 250.0 | 94.6 | — | — | — | — | — | — | — | — | — | — |
| Sodium choloride | — | — | 250.0 | 94.6 | — | — | — | — | — | — | — | — |
| Sucrose | — | — | — | — | 250.0 | 94.6 | — | — | — | — | — | — |
| Dextrose | — | — | — | — | — | — | 250.0 | 94.6 | — | — | — | — |
| Calcium sulfate | — | — | — | — | — | — | — | — | 250.0 | 94.6 | — | — |
| Fructose | — | — | — | — | — | — | — | — | — | — | 250.0 | 94.6 |
| % Potency (Amlodipine besylate): 90%~110% | — | 56.4 | — | 47.6 | — | 97.4 | — | 74.4 | — | 96.7 | — | 85.4 |
| % Potency (Dextromethorphan hydrobromide | — | 96.2 | — | 108.8 | — | 100.9 | — | 104.5 | — | 101.4 | — | 102.2 |
| monohydrate): 90%~110% | | | | | | | | | | | | |
| Any individual impurity (%): NMT 0.26% | — | 4.54 | — | 7.34 | — | 0.57 | — | 7.52 | — | 0.63 | — | 2.69 |
| Based on ICH Q3B (R2) identification thresholds | | | | | | | | | | | | |
| Total impurity (%): NMT 1.00% | — | 5.37 | — | 9.44 | — | 0.67 | — | 10.15 | — | 0.63 | — | 4.46 |

| | |
|---|---|
| Sample | Sample |
| A24 | A25 |

| | mg | % | mg | % |
|---|---|---|---|---|
| Amlodipine besylate | 6.9 | 2.6 | 6.9 | 2.6 |
| Dextromethorphan hydrobromide monohydrate | 7.5 | 2.8 | 7.5 | 2.8 |
| Dextrin | 250.0 | 94.6 | — | — |
| Kaolin | — | — | 250.0 | 94.6 |
| % Potency (Amlodipine besylate): 90%~110% | — | 98.8 | — | 73.9 |
| % Potency (Dextromethorphan hydrobromide monohydrate): 90%~110% | — | 101.3 | — | 90.3 |
| Any individual impurity (%): NMT 0.26% | — | N.D. | — | 0.5 |
| Based on ICH Q3B (R2) identification thresholds | | | | |
| Total impurity (%): NMT 1.00% | — | N.D. | — | 0.5 |

TABLE 17

| | Sample | | Sample | | Sample | | Sample | | Sample | | Sample | |
|---|---|---|---|---|---|---|---|---|---|---|---|---|
| | A26 | | A27 | | A28 | | A29 | | A30 | | A31 | |
| | mg | % | mg | % | mg | % | mg | % | mg | % | mg | % |
| Amlodipine | 2.5 | — | 2.5 | — | 5.0 | — | 5.0 | — | 10.0 | — | 12.35 | — |
| Dextromethorphan | 22 | — | 7.3 | — | 5.5 | — | 1.8 | — | 1.8 | — | 1.9 | — |
| Amlodipine besylate | 3.5 | 1.5 | 3.5 | 1.5 | 6.9 | 3.0 | 6.9 | 3.0 | 13.9 | 6.0 | 17.1 | 7.5 |
| Dextromethorphan hydrobromide monohydrate | 30.0 | 13.0 | 10.0 | 4.3 | 7.5 | 3.3 | 2.5 | 1.1 | 2.5 | 1.1 | 2.6 | 1.1 |
| Microcrystalline cellulose | 94.5 | 41.1 | 114.5 | 49.8 | 113.6 | 49.4 | 118.6 | 51.5 | 111.6 | 48.5 | 108.3 | 47.1 |
| Pregelatinized starch | 100.0 | 43.5 | 100.0 | 43.5 | 100 | 43.5 | 100 | 43.5 | 100.0 | 43.5 | 100.0 | 43.5 |
| Magnesium stearate | 1.0 | 0.4 | 1.0 | 0.4 | 1.0 | 0.4 | 1.0 | 0.4 | 1.0 | 0.4 | 1.0 | 0.4 |
| Colloidal silicon dioxide | 1.0 | 0.4 | 1.0 | 0.4 | 1.0 | 0.4 | 1.0 | 0.4 | 1.0 | 0.4 | 1.0 | 0.4 |
| Total | 230.0 | 100.0 | 230.0 | 100.0 | 230.00 | 100.0 | 230.0 | 100.0 | 230.0 | 100.0 | 230.0 | 100.0 |

| | | | | | | |
|---|---|---|---|---|---|---|
| Amlodipine/Dextromethorphan | 0.11 | 0.34 | 0.91 | 2.78 | 5.55 | 6.50 |

Assay

| | Sample | Sample | Sample | Sample | Sample | Sample |
|---|---|---|---|---|---|---|
| | A26 | A27 | A28 | A29 | A30 | A31 |
| % Potency (Amlodipine besylate): 90%~110% | 100.9 | 100.6 | 98.7 | 96.4 | 97.9 | 100.0 |
| % Potency (Dextromethorphan hydrobromide monohydrate): 90%~110% | 100.1 | 105.3 | 101.3 | 108.6 | 107.1 | 101.7 |
| Any individual impurity (%): | N.D. | N.D. | N.D. | N.D. | 0.04 | N.D. |
| Total impurity (%): NMT 1.00% | N.D. | N.D. | N.D. | N.D. | 0.04 | N.D. |
| Any individual impurity NMT (%): | 0.20 | 0.20 | 0.27 | 0.29 | 0.20 | 0.20 |
| Based on ICH Q3B (R2) identification thresholds | | | | | | |

TABLE 18
Pregelatinized starch as disintegrant agent, Mannitol as diluent agent

| | | Sample | | Sample | | Sample | | Sample | |
|---|---|---|---|---|---|---|---|---|---|
| | | A32 | | A33 | | A34 | | A35 | |
| Ingredient | Function | mg | % | mg | % | mg | % | mg | % |
| Amlodipine besylate | Active | 2.08 | 0.1 | 6.94 | 0.9 | 3.47 | 4.3 | 31.23 | 20.8 |
| Dextromethorphan hydrobromide monohydrate | Active | 1.58 | 0.1 | 7.88 | 1.1 | 2.63 | 3.3 | 30.7 | 20.5 |
| Mannitol | Diluent | 14.34 | 1.0 | 354.18 | 47.2 | 41.26 | 51.6 | 83.87 | 55.9 |
| Pregelatinized starch | Disintegrant | 1470 | 98.0 | 375 | 50.0 | 32 | 40.0 | 3 | 2.0 |
| Magnesium stearate | Lubricant | 6 | 0.4 | 3 | 0.4 | 0.32 | 0.4 | 0.6 | 0.4 |
| Colloidal silicon dioxide | Glidant | 6 | 0.4 | 3 | 0.4 | 0.32 | 0.4 | 0.6 | 0.4 |
| Total | | 1500 | 100 | 750 | 100.0 | 80 | 100 | 150 | 100 |

Assay

| | Sample A32 | Sample A33 | Sample A34 | Sample A35 |
|---|---|---|---|---|
| % Potency (Amlodipine besylate): 90%~110% | 101.8 | 94.9 | 95.2 | 96.9 |
| % Potency (Dextromethorphan hydrobromide monohydrate): 90%~110% | 94.9 | 98.7 | 100.0 | 99.4 |
| Any individual impurity (%): | 0.24 | 0.08 | 0.10 | 0.13. |
| Total impurity (%): NMT 1.00% | 0.29 | 0.08 | 0.19 | 0.13. |
| Any individual impurity NMT (%): | 0.50 | 0.25 | 0.50 | 0.20 |
| Based on ICH Q3B (R2) identification thresholds | | | | |

TABLE 19
Pregelatinized starch as disintegrant agent, Microcrystalline cellulose as diluent agent

| | | Sample | | Sample | | Sample | | Sample | |
|---|---|---|---|---|---|---|---|---|---|
| | | A36 | | A37 | | A38 | | A39 | |
| Ingredient | Function | mg | % | mg | % | mg | % | mg | % |
| Amlodipine besylate | Active | 2.08 | 0.1 | 6.94 | 0.9 | 3.47 | 4.3 | 31.23 | 20.8 |
| Dextromethorphan hydrobromide monohydrate | Active | 1.58 | 0.1 | 7.88 | 1.1 | 2.63 | 3.3 | 30.7 | 20.5 |
| Microcrystalline cellulose | Disintegrant/ | 14.34 | 1.0 | 354.18 | 47.2 | 41.26 | 51.6 | 83.87 | 55.9 |
| | Diluent | | | | | | | | |
| Pregelatinized starch | Disintegrant/ | 1470 | 98.0 | 375 | 50.0 | 32 | 40.0 | 3 | 2.0 |
| | Diluent t | | | | | | | | |
| Magnesium stearate | Lubricant | 6 | 0.4 | 3 | 0.4 | 0.32 | 0.4 | 0.6 | 0.4 |
| Colloidal silicon dioxide | Glidant | 6 | 0.4 | 3 | 0.4 | 0.32 | 0.4 | 0.6 | 0.4 |
| Total | | 1500 | 100 | 750 | 100.0 | 80 | 100 | 150 | 100 |

Assay

| | Sample A36 | Sample A37 | Sample A38 | Sample A39 |
|---|---|---|---|---|
| % Potency (Amlodipine besylate): 90%~110% | 103.4 | 94.9 | 95.5 | 98.0 |
| % Potency (Dextromethorphan hydrobromide monohydrate): 90%~110% | 97.6 | 98.7 | 100.4 | 98.9 |
| Any individual impurity (%): | 0.23 | 0.08 | N.D. | N.D. |
| Total impurity (%): NMT 1.00% | 0.23 | 0.08 | N.D. | N.D. |
| Any individual impurity NMT (%): | 0.50 | 0.25 | 0.50 | 0.20 |

TABLE 20
Microcrystalline cellulose as distintegrant agent, Mannitol as diluent agent

| | | Sample | | Sample | | Sample | | Sample | |
|---|---|---|---|---|---|---|---|---|---|
| | | A40 | | A41 | | A42 | | A43 | |
| Ingredient | Function | mg | % | mg | % | mg | % | mg | % |
| Amlodipine besylate | Active | 2.08 | 0.1 | 6.94 | 0.9 | 3.47 | 4.3 | 31.23 | 20.8 |
| Dextromethorphan hydrobromide monohydrate | Active | 1.58 | 0.1 | 7.88 | 1.1 | 2.63 | 3.3 | 30.7 | 20.5 |
| Mannitol | Diluent | 14.34 | 1.0 | 354.18 | 47.2 | 41.26 | 51.6 | 83.87 | 55.9 |
| Microcrystalline cellulose | Disintegrant | 1470 | 98.0 | 375 | 50.0 | 32 | 40.0 | 3 | 2.0 |
| Magnesium stearate | Lubricant | 6 | 0.4 | 3 | 0.4 | 0.32 | 0.4 | 0.6 | 0.4 |
| Colloidal silicon dioxide | Glidant | 6 | 0.4 | 3 | 0.4 | 0.32 | 0.4 | 0.6 | 0.4 |
| Total | | 1500 | 100 | 750 | 100.0 | 80 | 100 | 150 | 100 |

Assay

| | Sample A40 | Sample A41 | Sample A42 | Sample A43 |
|---|---|---|---|---|
| % Potency (Amlodipine besylate): 90%~110% | 100.3 | 95.0 | 91.8 | 97.5 |
| % Potency (Dextromethorphan hydrobromide monohydrate): 90%~110% | 105.8 | 103.0 | 102.1 | 98.8 |
| Any individual impurity (%): | 0.05 | 0.03 | 0.34 | 0.15 |
| Total impurity (%): NMT 1.00% | 0.05 | 0.03 | 0.34 | 0.15 |
| Any individual impurity NMT (%): | 0.50 | 0.25 | 0.50 | 0.20 |
| Based on ICH Q3B (R2) identification thresholds | | | | |

TABLE 21
Weight ranging from 80 to 1500 mg.

| | | Sample | | Sample | | Sample | |
|---|---|---|---|---|---|---|---|
| | | A44 | | A45 | | A46 | |
| Ingredient | Function | mg | % | mg | % | mg | % |
| Amlodipine besylate | Active | 2.08 | 0.1 | 6.94 | 0.9 | 3.47 | 4.3 |
| Dextromethorphan hydrobromide monohydrate | Active | 1.58 | 0.1 | 7.88 | 1.1 | 2.63 | 3.3 |
| Microcrystalline cellulose | Diluent | 14.34 | 1.0 | 354.18 | 47.2 | 41.26 | 51.6 |
| Pregelatinized starch | Disintegratit | 1470 | 98.0 | 375 | 50.0 | 32 | 40.0 |
| Magnesium stearate | Lubricant | 6 | 0.4 | 3 | 0.4 | 0.32 | 0.4 |
| Colloidal silicon dioxide | Glidant | 6 | 0.4 | 3 | 0.4 | 0.32 | 0.4 |
| Total | | 1500 | 100 | 750 | 100.0 | 80 | 100 |

Assay

| | Sample A44 | Sample A45 | Sample A46 |
|---|---|---|---|
| % Potency (Amlodipine besylate): 90%~110% | 103.4 | 94.9 | 95.5 |
| % Potency (Dextromethorphan hydrobromide monohydrate): 90%~110% | 97.6 | 98.7 | 100.4 |
| Any individual impurity (%): | 0.23 | 0.08 | N.D. |
| Total impurity (%): NMT 1.00% | 0.23 | 0.08 | N.D. |
| Any individual impurity NMT (%): | 0.50 | 0.25 | 0.50 |
| Based on ICH Q3B (R2) identification thresholds | | | |

TABLE 22

| | | Sample | | Sample | | Sample | | Sample | |
|---|---|---|---|---|---|---|---|---|---|
| | | A47 | | A48 | | A49 | | A50 | |
| Ingredient | Function | mg | % | mg | % | mg | % | mg | % |
| Amlodipine besylate | Active | 2.08 | 0.1 | 31.23 | 20.8 | 3.47 | 0.7 | 3.47 | 4.3 |
| Dextromethorphan hydrobromide monohydrate | Active | 1.58 | 0.1 | 30.7 | 20.5 | 2.63 | 0.5 | 2.63 | 3.3 |
| Microcrystalline cellulose | Disintegrant/ | 14.34 | 1.0 | 83.87 | 55.9 | 479.9 | 96.0 | 41.2626 | 51.6 |
| | Diluent | | | | | | | | |
| Pregelatinized starch | Disintegrant/ | 1470 | 98.0 | 3 | 2.0 | 10 | 2.0 | 32 | 40.0 |
| | Diluent | | | | | | | | |
| Magnesium stearate | Lubricant | 6 | 0.4 | 0.6 | 0.4 | 2 | 0.4 | 0.32 | 0.4 |
| Colloidal silicon dioxide | Glidant | 6 | 0.4 | 0.6 | 0.4 | 2 | 0.4 | 0.32 | 0.4 |
| Total | | 1500 | 100 | 150 | 100 | 500 | 100 | 80 | 100 |

Assay

| | Sample A47 | Sample A48 | Sample A49 | Sample A50 |
|---|---|---|---|---|
| % Potency (Amlodipine besylate): 90%~110% | 103.4 | 98.0 | 94.1 | 95.5 |
| % Potency (Dextromethorphan hydrobromide monohydrate): 90%~110% | 97.6 | 98.9 | 104.1 | 100.4 |
| Any individual impurity (%): | 0.23 | N.D. | 0.05 | N.D. |
| Total impurity (%): NMT 1.00% | 0.23 | N.D. | 0.05 | N.D. |
| Any individual impurity NMT (%): | 0.50 | 0.20 | 0.50 | 0.50 |
| Based on ICH Q3B (R2) identification thresholds | | | | |

1. A pharmaceutical composition in a solid oral form comprising: amlodipine or a pharmaceutically acceptable salt thereof, dextromethorphan or a pharmaceutically acceptable salt thereof, and one or more pharmaceutically acceptable disintegrants or diluents selected from the group consisting of pregelatinized starch, sodium starch glycolate, microcrystalline cellulose, low-substituted hydroxypropyl cellulose, corn starch, carboxymethylcellulose sodium, croscarmellose sodium, ethylcellulose, talc, dextrin, mannitol, and any combination thereof.

2. The pharmaceutical composition according to claim 1, in a unit dosage form of a tablet or a capsule.

3. The pharmaceutical composition according to claim 2, wherein the total weight of the unit dosage is 80-1500 mg.

4. The pharmaceutical composition according to claim 1, wherein the amlodipine is in an amount of 0.1-30% w/w of the pharmaceutical composition.

5. The pharmaceutical composition according to claim 1, wherein the dextromethorphan is in an amount of 0.1-30% w/w of the pharmaceutical composition.

6. The pharmaceutical composition according to claim 5, wherein the dextromethorphan is in an amount of 0.1-10% w/w of the pharmaceutical composition.

7. The pharmaceutical composition according to claim 1, wherein weight ratios of amlodipine versus dextromethorphan are within 0.1 to 6.5.

8. The pharmaceutical composition according to claim 1, wherein weight ratios of amlodipine versus dextromethorphan are within 0.1 to 4.

9. The pharmaceutical composition according to claim 1, further comprising 0.1-3% w/w of a lubricant.

10. The pharmaceutical composition according to claim 9, wherein the lubricant is magnesium stearate.

11. The pharmaceutical composition according to claim 1, further comprising 0.1-3% w/w of a glidant.

12. The pharmaceutical composition according to claim 11, wherein the glidant is colloidal silicon dioxide.

13. The pharmaceutical composition according to claim 1, wherein the distintegrants or diluents are in an amount to 2-98% w/w of the pharmaceutical composition.

14. The pharmaceutical composition according to claim 1, comprising 0.1-15% w/w of amlodipine, 0.1-15% w/w of dextromethorphan, 3-60% w/w of pregelatinized starch, and 30-90% w/w microcrystalline cellulose.

15. The pharmaceutical composition according to claim 1, wherein amlodipine is in the form of the besylate salt thereof.

16. The pharmaceutical composition according to claim 1, wherein dextromethorphan is in the form of the hydrobromide salt thereof.

17. Use of the pharmaceutical composition according to claim 1, for preparing a medicament for treating hypertension.
